(19) United States
(12) Patent Application Publication
(10) Pub. No.: US 2025/0098978 A1
(43) Pub. Date: Mar. 27, 2025

(54) MAGNETIC RESONANCE DIAGNOSTIC APPARATUS

(71) Applicant: FUJIFILM Corporation, Tokyo (JP)

(72) Inventor: Hiroki MORIYA, Chiba (JP)

(73) Assignee: FUJIFILM Corporation, Tokyo (JP)

(21) Appl. No.: 18/890,766

(22) Filed: Sep. 20, 2024

(30) Foreign Application Priority Data
Sep. 25, 2023 (JP) 2023-161543

(51) Int. Cl. A61B 5/055; A61B 5/00

(52) U.S. Cl. CPC A61B 5/055; A61B 5/704

(57) ABSTRACT

Provided is a magnetic resonance diagnostic apparatus capable of eliminating a cable entanglement problem. In a state in which a reception-side connector is connected to an examination table-side connector, a reception-side cable is stored in a cable storage part by pulling in an examination table-side cable via a cable drive mechanism, and the reception-side cable is fed out from the cable storage part by feeding out the examination table-side cable.

### CROSS-REFERENCE TO RELATED APPLICATION

The present application claims priority under 35 U.S.C § 119(a) to Japanese Patent Application No. 2023-161543 filed on Sep. 25, 2023, which is hereby expressly incorporated by reference, in its entirety, into the present application.

### BACKGROUND OF THE INVENTION

### 1. Field of the Invention

The present invention relates to a magnetic resonance diagnostic apparatus, and particularly relates to a technology of storing a reception-side cable of a reception coil attached to a subject in an examination table.

### 2. Description of the Related Art

JP2020-146115A discloses a magnetic resonance imaging (MRI) apparatus in which a reception coil cable connected to a local use reception coil is connected to a connector of an examination table-side coil cable, and the examination table-side coil cable is pulled out from and wound up into a storage part of an examination table as necessary.

According to JP2020-146115A, before imaging is started, the examination table-side coil cable can be pulled out from the storage part, in a case in which the imaging is started, the examination table-side coil cable cannot be pulled from the storage part and wound up into the storage part, and in a case in which the imaging is ended, the examination table-side coil cable can be wound up into the storage part.

### SUMMARY OF THE INVENTION

However, the MRI apparatus of JP2020-146115A has a problem in that, in a case in which the examination table (top plate) is moved into a bore, the reception coil cable (corresponding to a first cable) gets entangled in a gap between a gantry and the top plate (hereinafter, this problem is referred to as a “cable entanglement problem”).

The present invention has been made in view of such circumstances, and an object of the present invention is to provide a magnetic resonance diagnostic apparatus capable of eliminating a cable entanglement problem.

A first aspect of the present invention provides a magnetic resonance diagnostic apparatus comprising: a reception coil that is attached to a subject to be imaged, and receives a magnetic resonance signal generated in the subject; a first cable that is connected to the reception coil and has a first connector; an examination table on which the subject is placed; a cable storage part that is provided in the examination table and stores the first cable; a second cable that is stored in the cable storage part and has a second connector connected to the first connector; and a cable drive mechanism that pulls in or feeds out the second cable in the cable storage part, in which, in a state in which the first connector is connected to the second connector, the first cable is stored in the cable storage part by pulling in the second cable via the cable drive mechanism, and the first cable is fed out from the cable storage part by feeding out the second cable.

In the magnetic resonance diagnostic apparatus according to the first aspect of the present invention, for example, before imaging is started, the first connector of the first cable and the second connector of the second cable are connected to connect the first cable and the second cable. In a case in which a top plate is moved into a bore, the first cable (an extra length thereof) is stored in the cable storage part by pulling in the second cable via the cable drive mechanism. As a result, it is possible to prevent the first cable from being entangled in a gap between a gantry and the top plate in a case in which the top plate is moved into the bore. Therefore, it is possible to eliminate the cable entanglement problem. The imaging (inspection) is executed in a state in which the first cable (the extra length thereof) is stored in the cable storage part. After the imaging is ended, in a case in which the top plate is moved out of the bore, the second cable is fed out by the cable drive mechanism to feed out the first cable (the extra length thereof) from the cable storage part. As a result, it is possible to prevent excessive tension from being applied to the first cable in a case in which the top plate is moved out of the bore.

Preferably, a second aspect of the present invention provides the magnetic resonance diagnostic apparatus according to the first aspect, which further comprises: an operation unit that is operable by an operator, in which the cable drive mechanism is driven in a case in which the operation unit is operated.

Preferably, a third aspect of the present invention provides the magnetic resonance diagnostic apparatus according to the second aspect, which further comprises: an examination table moving unit that moves the examination table to an imaging position, in which the examination table moving unit is driven by the operation of the operation unit.

Preferably, a fourth aspect of the present invention provides the magnetic resonance diagnostic apparatus according to the third aspect, in which the cable drive mechanism and the examination table moving unit are interlocked with each other by a single operation of the operation unit.

Preferably, a fifth aspect of the present invention provides the magnetic resonance diagnostic apparatus according to the fourth aspect, in which, in a case in which the operation unit is operated, an operation of storing the first cable in the cable storage part via the cable drive mechanism and an operation of moving the examination table to the imaging position are performed.

Preferably, a sixth aspect of the present invention provides the magnetic resonance diagnostic apparatus according to the first or second aspect, in which the cable storage part is provided on an outer peripheral part of the examination table.

Preferably, a seventh aspect of the present invention provides the magnetic resonance diagnostic apparatus according to the first or second aspect, which further comprises: a motor that stores the first cable in the cable storage part, in which the first cable is stored in the cable storage part by a driving force of the motor.

Preferably, an eighth aspect of the present invention provides the magnetic resonance diagnostic apparatus according to the first or second aspect, in which the second connector includes a cable fixing member that is capable of fixing a cable member different from the first cable.

Preferably, a ninth aspect of the present invention provides the magnetic resonance diagnostic apparatus according to the seventh aspect, which further comprises: a cable storage operation stop part that forcibly stops a storage operation of storing the first cable in the cable storage part.

Preferably, a tenth aspect of the present invention provides the magnetic resonance diagnostic apparatus according to the second aspect, in which the operation unit includes a handle that manually drives the cable drive mechanism.

Preferably, an eleventh aspect of the present invention provides the magnetic resonance diagnostic apparatus according to the third aspect, in which the first cable is automatically fed out from the cable storage part in conjunction with an operation of pulling out the examination table from the imaging position via the operation unit.

Preferably, a twelfth aspect of the present invention provides the magnetic resonance diagnostic apparatus according to the third aspect, in which after an examination is ended, the examination table is automatically pulled out from the imaging position, and the first cable is automatically fed out from the cable storage part.

According to the present invention, it is possible to eliminate the cable entanglement problem.

### BRIEF DESCRIPTION OF THE DRAWINGS

FIG. 1 is a perspective view of an MRI apparatus.

FIG. 2 is a schematic diagram schematically showing a top plate drive mechanism.

FIG. 3 is a schematic diagram in a case in which a top plate is moved to an imaging position.

FIG. 4 is a top view showing a configuration of a cable drive mechanism.

FIG. 5 is a side view of the cable drive mechanism shown in FIG. 4.

FIG. 6 is a flowchart showing an imaging procedure by the MRI apparatus according to an embodiment.

FIG. 7 is a schematic diagram showing a front side of an examination table-side connector.

FIGS. 8A and 8B are explanatory views showing a cable fixing member.

FIG. 9 is a top view of a cable drive mechanism having a handle.

FIG. 10 is a side view of the cable drive mechanism shown in FIG. 9.

FIG. 11 is a top view showing a state in which entry of the examination table-side connector into a cable storage part is restricted by a lock mechanism.

FIG. 12 is a side view of FIG. 11.

FIG. 13 is a top view showing a state in which the entry of the examination table-side connector into the cable storage part is released by the lock mechanism.

FIG. 14 is a side view of FIG. 13.

### DESCRIPTION OF THE PREFERRED EMBODIMENTS

Hereinafter, an embodiment of a magnetic resonance diagnostic apparatus according to the present invention will be described with reference to the accompanying drawings.

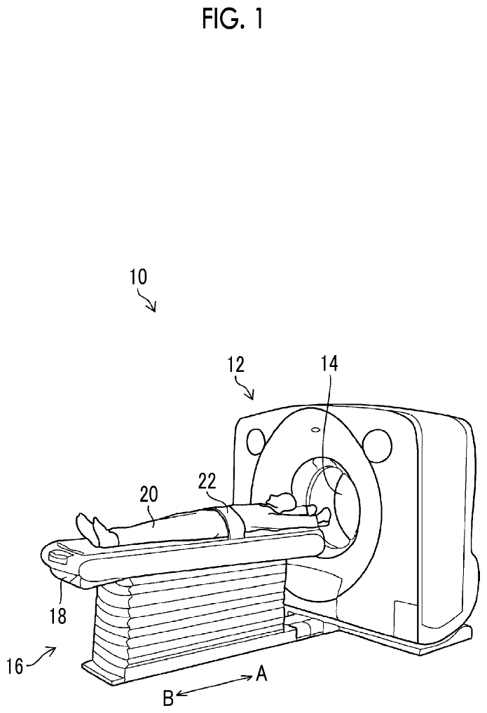

FIG. 1 is a perspective view showing an example of an MRI apparatus 10 to which a magnetic resonance diagnostic apparatus according to the embodiment of the present invention is applied.

### MRI Apparatus

As shown in FIG. 1, the MRI apparatus 10 comprises a gantry 12 and an examination table 16. The gantry 12 has a bore 14 which is an examination (imaging) space on a cylinder. In addition, the gantry 12 has an MRI magnet and various other coils disposed therein.

The examination table 16 is installed to face the bore 14 on a front side of the gantry 12. The examination table 16 includes a top plate 18 on an upper part. An examination site of a subject 20 to be imaged, which is placed on the top plate 18, is set to a center of a static magnetic field in the bore 14 by moving the top plate 18 within the bore 14.

An radio frequency (RF) coil 22 for receiving a nuclear magnetic resonance (NMR) signal generated in the subject 20 is attached to the examination site of the subject 20. In the present example, an abdomen of the subject 20 is the examination site, and the RF coil 22 is attached to the abdomen. The RF coil 22 is an example of a reception coil according to the present invention.

A reception-side cable, which will be described below, is connected to the RF coil 22. A reception-side connector is connected to an end part of the reception-side cable, and the reception-side connector is connected to an examination table-side connector of an examination table-side cable. As a result, the reception-side cable and the examination table-side cable are connected via their respective connectors.

The examination table-side cable is stored in a cable storage part of the examination table 16. A reception signal of the RF coil 22 is transmitted to a signal processing unit via the reception-side cable and the examination table-side cable, and is processed by the signal processing unit and converted into an image signal. The image signal is displayed as an MRI image by an MRI display unit.

### Top Plate Drive Mechanism

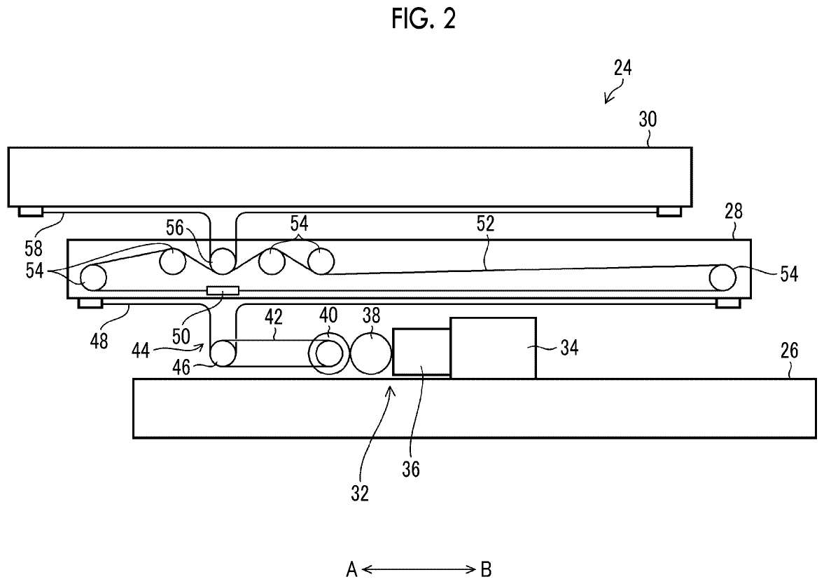

FIG. 2 is a schematic diagram schematically showing a structure of a top plate drive mechanism 24 for moving the top plate 18. Hereinafter, a configuration and an operation of the top plate drive mechanism 24 will be briefly described.

As shown in FIG. 2, the top plate drive mechanism 24 comprises an upper frame 26, a lower top plate 28, an upper top plate 30, and a driving unit 32. The top plate 18 is formed of the lower top plate 28 and the upper top plate 30. The top plate drive mechanism 24 moves the top plate 18 of the examination table 16 to an imaging position, and is an example of an examination table moving unit according to the present invention.

The upper frame 26 is fixed to an upper part of the examination table body (not shown). Among components of the driving unit 32, a DC motor 34, a reduction gear 36, an electromagnetic clutch 38, a pulley 40, a lower top plate drive belt 42, and a pulley 46 of a drive unit 44 are mounted on the upper frame 26.

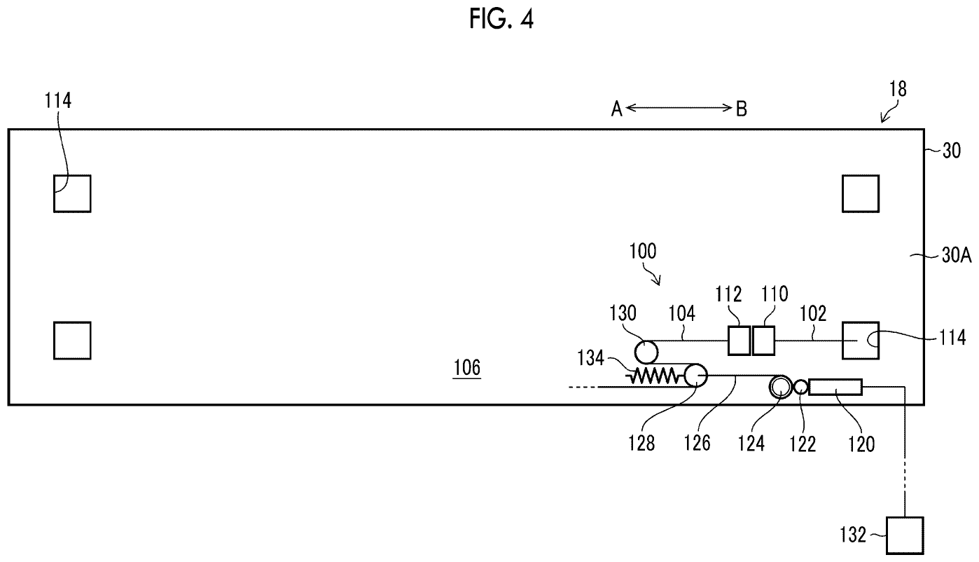

The DC motor 34 is driven by a top plate advancement operation and a top plate retraction operation by a top plate operation unit 132 (see FIG. 4) described below. A rotational force of the DC motor 34 is reduced by the reduction gear 36 and then transmitted to the pulley 40 via the electromagnetic clutch 38. The endless lower top plate drive belt 42 is stretched between the pulley 40 and the pulley 46, and in a case in which the pulley 40 is rotated, the lower top plate drive belt 42 is driven (circumferentially moved) by the rotation of the pulley 40, causing the pulley 46 to rotate.

The electromagnetic clutch 38 has a function of transmitting a driving force from the reduction gear 36 to the pulley 40 and a function of forcibly interrupting the power transmitted from the reduction gear 36 to the pulley 40 by an operation of stopping the top plate via the top plate operation unit 132.

The lower top plate drive belt 42 is disposed along movement directions of the lower top plate 28 indicated by arrows A and B. Here, the arrow A indicates a direction in which the lower top plate 28 moves from an outside of the bore 14 to an inside of the bore 14. In addition, the arrow B indicates a direction in which the lower top plate 28 moves from the inside of the bore 14 to the outside of the bore 14. That is, the arrow A is a direction in which the top plate 18 moves from a retracted position (see FIG. 1) toward the imaging position, and the arrow B is a direction in which the top plate 18 moves from the imaging position toward the retracted position.

Returning to FIG. 2, a lower top plate drive belt 48 with ends is wound around the pulley 46. The lower top plate drive belt 48 is disposed on a lower surface side of the lower top plate 28 along a movement direction of the lower top plate 28, and both end parts thereof are fixed to a lower surface of the lower top plate 28. The lower top plate drive belt 48 is one of the components of the driving unit 32.

Here, in a case in which the top plate advancement operation is executed by the top plate operation unit 132, the DC motor 34 rotates in a forward direction and the pulley 46 (intermediate power transmission system is omitted) rotates in a clockwise direction. As a result, the lower top plate drive belt 48 on a right side in FIG. 2 is wound around the pulley 46, and the lower top plate drive belt 48 on a left side in FIG. 2 is fed out. As a result, the lower top plate 28 moves in the direction of the arrow A.

In addition, in a case in which the top plate retraction operation is executed by the top plate operation unit 132, the DC motor 34 rotates in a reverse direction and the pulley 46 rotates in a counterclockwise direction. As a result, the lower top plate drive belt 48 on the left side in FIG. 2 is wound around the pulley 46, and the lower top plate drive belt 48 on the left side in FIG. 2 is fed out. As a result, the lower top plate 28 moves in the direction of the arrow B.

The pulley 46 is provided with a fixed portion 50 that extends upward. The fixed portion 50 is fixed to an endless upper top plate drive belt 52. The fixed portion 50 is one of the components of the drive unit 44.

The upper top plate drive belt 52 is stretched around a plurality of pulleys 54 along a movement direction (the same as the movement direction of the lower top plate 28) of the upper top plate 30 inside the lower top plate 28. In a case in which the lower top plate 28 is moved as described above, the upper top plate drive belt 52 is driven (circumferentially moved) along the movement direction of the upper top plate 30 because a relative position between the pulley 46 and the fixed portion 50 is maintained. The upper top plate drive belt 52 is one of the components of the driving unit 32.

The upper top plate drive belt 52 is wound around a pulley 56 provided on a lower surface side of the upper top plate 30. Therefore, in a case in which the upper top plate drive belt 52 is driven, the pulley 56 is rotated.

In addition, an upper top plate drive belt 58 with ends is wound around the pulley 56. The upper top plate drive belt 58 is disposed on the lower surface side of the upper top plate 30 along the movement direction of the top plate 30, and both end parts thereof are fixed to a lower surface of the upper top plate 30. The upper top plate drive belt 58 is one of the components of the driving unit 32.

Here, in a case in which the top plate advancement operation is executed by the top plate operation unit 132, the DC motor 34 rotates in the forward direction and the pulley 56 (intermediate power transmission system is omitted) rotates in the clockwise direction. As a result, the upper top plate drive belt 58 on the right side in FIG. 2 is wound around the pulley 56, and the upper top plate drive belt 58 on the left side in FIG. 2 is fed out. As a result, the upper top plate 30 moves in the direction of the arrow A.

In addition, in a case in which the top plate retraction operation is executed by the top plate operation unit 132, the DC motor 34 rotates in a reverse direction and the pulley 56 rotates in a counterclockwise direction. As a result, the upper top plate drive belt 58 on the left side in FIG. 2 is wound around the pulley 56, and the upper top plate drive belt 58 on the right side in FIG. 2 is fed out. As a result, the upper top plate 30 moves in the direction of the arrow B.

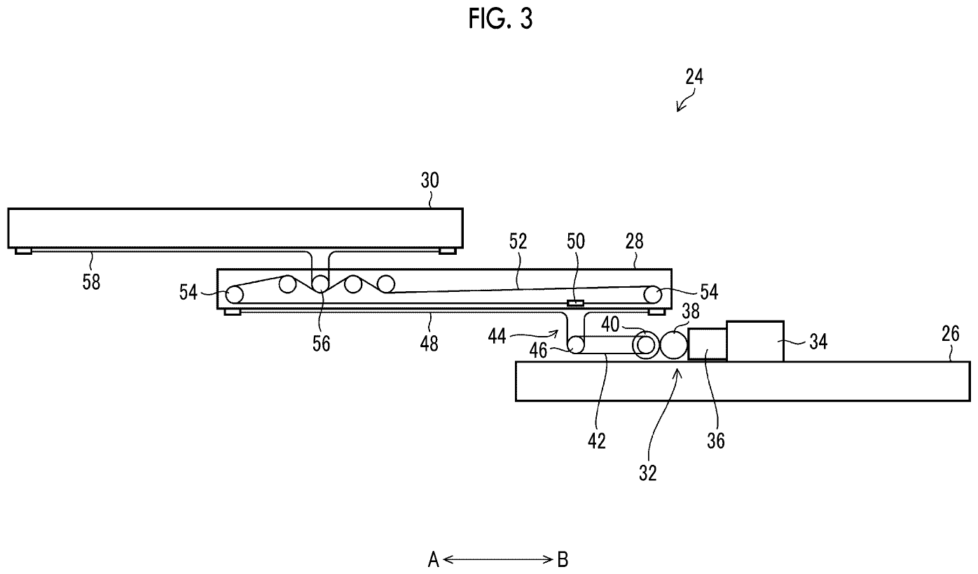

FIG. 3 is a schematic diagram showing a state in which the top plate 18 is moved to the imaging position. According to FIG. 3, the lower top plate 28 is moved in the direction of the arrow A by the power of the driving unit 32, and the upper top plate 30 is moved in the direction of the arrow A in accordance with the movement of the lower top plate 28 in the direction of the arrow A. As a result, the top plate 18 is moved to the imaging position in the bore 14.

In a case in which the lower top plate 28 is moved in the direction of the arrow B by the power of the driving unit 32, the upper top plate 30 is moved in the direction of the arrow B in accordance with the movement of the lower top plate 28 in the direction of the arrow B. As a result, the top plate 18 is moved to the retracted position (see FIG. 1) outside the bore 14.

As described above, with the top plate drive mechanism 24 of the present example, the top plate 18 can be moved between the retracted position and the imaging position by the power of one DC motor 34.

Incidentally, in the field of the MRI apparatus, there is a problem (cable entanglement problem) in that the reception-side cable connected to the RF coil gets entangled in a gap between the top plate and the gantry in a case in which the top plate is moved into the bore, which is the imaging position.

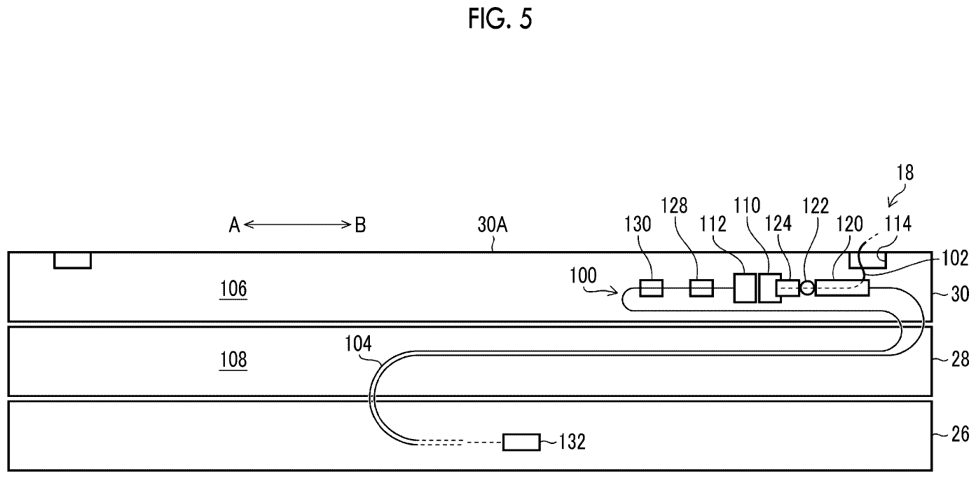

Therefore, the MRI apparatus 10 according to the embodiment has the following configuration in order to eliminate the above-described cable entanglement problem. Hereinafter, a configuration for eliminating the cable entanglement problem will be described with reference to FIGS. 4 and 5.

### Cable Drive Mechanism

FIG. 4 is a top view showing a configuration of the cable drive mechanism 100 installed on the examination table 16, and FIG. 5 is a side view of the cable drive mechanism 100 shown in FIG. 4.

As shown in FIGS. 4 and 5, the cable drive mechanism 100 is installed inside the upper top plate 30. In addition, a cable storage part 106 for storing the reception-side cable 102 and the examination table-side cable 104 is provided in the upper top plate 30, and a cable storage part 108 for storing the examination table-side cable 104 is provided in the lower top plate 28.

The cable drive mechanism 100 is an example of a cable drive mechanism according to the present invention. The reception-side cable 102 is an example of a first cable according to the present invention, and the examination table-side cable 104 is an example of a second cable according to the present invention. The cable storage part 106 is an example of a cable storage part according to the present invention.

In the examination table 16 of the present example, a part of an internal space of the upper top plate 30 is used as the cable storage part 106, and a part of an internal space of the lower top plate 28 is used as the cable storage part 108.

The cable storage part 106 and the cable storage part 108 are provided on an outer peripheral part of the examination table 16 so as not to affect the imaging. The outer peripheral part of the examination table 16 refers to an outer peripheral region that is present outside an examination site region of the subject 20 in a case in which the subject 20 is placed on the top plate 18.

One end of the reception-side cable 102 is connected to the RF coil 22 (see FIG. 1) as described above, and the other end of the reception-side cable 102 is connected to the reception-side connector 110. In addition, the examination table-side connector 112 is connected to one end of the examination table-side cable 104, and the other end of the examination table-side cable 104 is connected to the signal processing unit described above.

FIGS. 4 and 5 show a state in which the reception-side connector 110 and the examination table-side connector 112 are connected. As a result, the reception-side cable 102 and the examination table-side cable 104 are connected. As a result, the RF coil 22 and the signal processing unit are connected via the reception-side cable 102 and the examination table-side cable 104.

In addition, FIGS. 4 and 5 show a state in which the reception-side connector 110 and the examination table-side connector 112 are connected in the cable storage part 106, but connection work between the reception-side connector 110 and the examination table-side connector 112 is performed, for example, on an upper surface 30A of the upper top plate 30.

A cable hole 114 communicating with the cable storage part 106 is provided at a corner portion of the upper surface 30A of the upper top plate 30. The examination table-side cable 104 stored in the cable storage part 106 is pulled out by a predetermined length from the cable hole 114 with the examination table-side connector 112 taking the forefront. Then, the reception-side connector 110 is connected to the pulled-out examination table-side connector 112. Thereafter, the pulled-out examination table-side cable 104 and the reception-side cable 102 (the extra length thereof) are stored in the cable storage part 106 by the cable drive mechanism 100. This cable storage operation is performed in a case in which the top plate 18 is moved into the bore 14, as will be described below.

As shown in FIGS. 4 and 5, the cable drive mechanism 100 includes a motor 120, an electromagnetic clutch 122, a geared roller 124, a drive belt 126, a driven pulley 128, and a fixed pulley 130.

The examination table-side cable 104 is wound around the driven pulley 128 and the fixed pulley 130.

Specifically, the examination table-side cable 104 extends from the examination table-side connector 112 in the direction of the arrow A (a direction in which the top plate 18 moves from the retracted position toward the imaging position) and is wound around the fixed pulley 130. The examination table-side cable 104 extends from the fixed pulley 130 in the direction of the arrow B (a direction in which the top plate 18 moves from the imaging position toward the retracted position) and is wound around the driven pulley 128. The examination table-side cable 104 extends from the driven pulley 128 in the direction of the arrow A and is disposed in the cable storage part 108 from the cable storage part 106.

The motor 120 is driven by the top plate advancement operation via the top plate operation unit 132 described above. Therefore, in the present example, the cable drive mechanism 100 (the motor 120 thereof) and the top plate drive mechanism 24 (the DC motor 34 thereof) are interlocked with each other by the top plate advancement operation of the top plate operation unit 132. The motor 120 is an example of a motor according to the present invention.

The top plate operation unit 132 is an operation unit that can be operated by an operator. The top plate operation unit 132 may be provided in an existing touch panel type graphical user interface (GUI) switch provided on a front wall of the gantry 12, or may be provided in a hard push-button switch, a foot switch, or a manual handle provided in the periphery of the gantry 12 and the examination table 16. The top plate operation unit 132 is an example of an operation unit according to the present invention.

A rotational force of the motor 120 is transmitted to a gear of the geared roller 124 via a gear (not shown) of the electromagnetic clutch 122. The geared roller 124 and the driven pulley 128 are connected via the drive belt 126 with ends, and in a case in which the geared roller 124 is rotated, the drive belt 126 is wound around the geared roller 124 by the rotation of the geared roller 124. As a result, the driven pulley 128 is moved in the direction of the arrow B.

The driven pulley 128 is pre-biased in the direction of the arrow A by a biasing force of a spring 134. Therefore, the driven pulley 128 is moved in the direction of the arrow B against the biasing force of the spring 134.

In a case in which the driven pulley 128 is moved in the direction of the arrow B, the examination table-side cable 104 between the examination table-side connector 112 and the fixed pulley 130 is pulled in the direction of the arrow A. As a result, the reception-side cable 102 is stored in the cable storage part 106, and the extra length of the reception-side cable 102 disposed on the top plate 18 is stored in the cable storage part 106. The operation of storing the reception-side cable 102 is performed in conjunction with the movement of the top plate 18 in the direction of the arrow A.

Via the operation of storing the reception-side cable 102, the reception-side cable 102 disposed on the top plate 18 gradually takes off its slack and is finally in a state of no slack or in a slack state to a degree that does not cause the cable entanglement problem even in a case where the slack is present.

As a result, it is possible to eliminate a problem (cable entanglement problem) in that the reception-side cable 102 gets entangled in a gap between the gantry 12 and the top plate 18 in a case in which the top plate 18 is moved (moves in the direction of the arrow A) into the bore 14.

The electromagnetic clutch 122 is driven by the top plate retraction operation via the top plate operation unit 132. The electromagnetic clutch 122 is released by this top plate retraction operation, allowing the geared roller 124 to rotate in a rewinding direction. The top plate 18 starts moving (moving in the direction of the arrow B) from the imaging position toward the retracted position (position in FIG. 1) by the top plate retraction operation via the top plate operation unit 132.

In a case in which the electromagnetic clutch 122 is released, the geared roller 124 is released from restriction in the rewinding direction via the electromagnetic clutch 122, and is in a free state of rotation in the rewinding direction. As a result, the driven pulley 128 is moved in the direction of the arrow A by the biasing force of the spring 134, and the drive belt 126 that has been wound around the geared roller 124 is rewound. Then, the examination table-side cable 104 between the examination table-side connector 112 and the fixed pulley 130 is fed out in the direction of the arrow B. As a result, the reception-side cable 102 (the extra length thereof) that has been stored in the cable storage part 106 is fed out from the cable storage part 106 without slack via the cable hole 114. The operation of feeding out the reception-side cable 102 is performed in conjunction with the movement of the top plate 18 in the direction of the arrow B.

As a result, it is possible to eliminate a problem (cable entanglement problem) in that the extra length of the reception-side cable 102 gets entangled in a gap between the gantry 12 and the top plate 18 in a case in which the top plate 18 is moved (moves in the direction of the arrow B) out of the bore 14. In addition, it is possible to prevent excessive tension from being applied to the reception-side cable 102 in a case in which the top plate 18 is moved out of the bore 14.

### Imaging Procedure

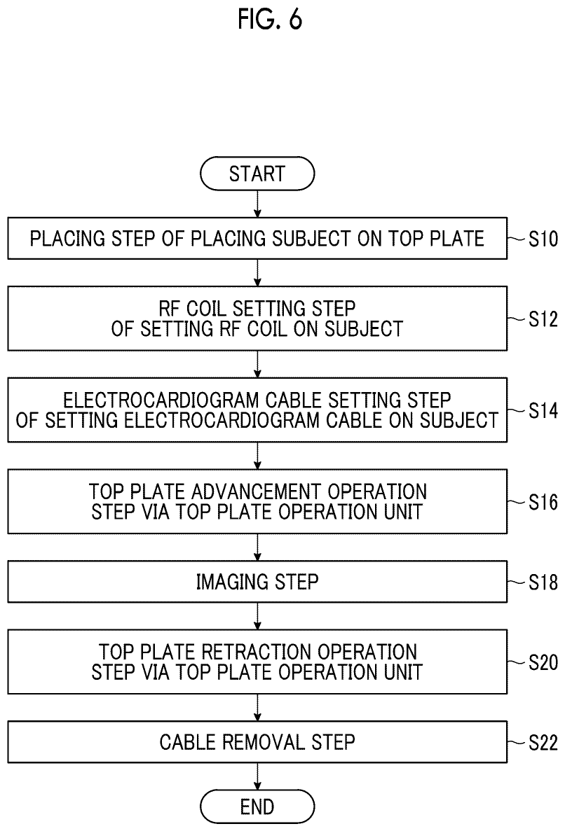

Hereinafter, an example of an imaging procedure of the MRI apparatus 10 according to the embodiment will be described with reference to FIG. 6. FIG. 6 is a flowchart showing the imaging procedure.

### Step S10: Placing Step

First, the subject 20 is placed on the top plate 18.

### Step S12: RF Coil Setting Step

Next, the RF coil 22 is attached to the abdomen of the subject 20. After that, the reception-side connector 110 of the reception-side cable 102 is connected to the examination table-side connector 112 of the examination table-side cable 104.

### Step S14: Electrocardiogram Cable Setting Step

Next, an electrocardiogram cable is attached to the subject 20. The attachment of the electrocardiogram cable is not required in some cases. Therefore, this step is performed only as necessary.

### Step S16: Top Plate Advancement Operation Step

Next, the top plate operation unit 132 is operated to perform the top plate advancement operation. The top plate drive mechanism 24 is driven by the top plate advancement operation, and the top plate 18 is moved from the retracted position toward the imaging position (from the outside of the bore 14 to the inside of the bore 14) in the direction of the arrow A.

In this case, the cable drive mechanism 100 is also driven, and the reception-side cable 102 is stored in the cable storage part 106. This storage operation is continued, for example, until the top plate 18 reaches the imaging position, and is stopped when the top plate 18 reaches the imaging position.

### Step S18: Imaging Step

Next, the subject 20 is imaged by the MRI apparatus 10.

### Step S20: Top Plate Retraction Operation Step

Next, the top plate operation unit 132 is operated to perform the top plate retraction operation. The top plate drive mechanism 24 is driven by the top plate retraction operation, and the top plate 18 is moved from the imaging position toward the retracted position (from the inside of the bore 14 to the outside of the bore 14) in the direction of the arrow B.

In this case, the cable drive mechanism 100 is driven, and the reception-side cable 102 (the extra length thereof) is fed out from the cable storage part 106 to the outside via the cable hole 114. This operation is continued, for example, until the top plate 18 reaches the retracted position, and is stopped when the top plate 18 reaches the retracted position.

### Step S22: Cable Removal Step

Next, the RF coil 22 and the electrocardiogram cable are removed from the subject 20. With the above, the image diagnosis by the MRI apparatus 10 is ended.

With the MRI apparatus 10 according to the embodiment as described above, before the imaging is started, the reception-side connector 110 and the examination table-side connector 112 are connected and the reception-side cable 102 and the examination table-side cable 104 are connected. In a case in which the top plate 18 is moved into the bore 14, the reception-side cable 102 (the extra length thereof) is stored in the cable storage part 106 by pulling in the examination table-side cable 104 via the cable drive mechanism 100.

As a result, it is possible to prevent the reception-side cable 102 from being entangled in a gap between the gantry 12 and the top plate 18 in a case in which the top plate 18 is moved into the bore 14. Therefore, it is possible to eliminate the cable entanglement problem.

The imaging is executed in a state in which the reception-side cable 102 (the extra length thereof) is stored in the cable storage part 106.

After the imaging is ended, in a case in which the top plate 18 is moved out of the bore 14, the examination table-side cable 104 is fed out by the cable drive mechanism 100 to feed out the reception-side cable 102 (the extra length thereof) from the cable storage part 106.

As a result, it is possible to prevent the reception-side cable 102 from being entangled in a gap between the gantry 12 and the top plate 18 in a case in which the top plate 18 is moved out of the bore 14. Therefore, it is possible to eliminate the cable entanglement problem. Further, it is possible to prevent excessive tension from being applied to the reception-side cable 102.

Therefore, the MRI apparatus 10 according to the embodiment adopts the configuration in which, in a state in which the reception-side connector 110 is connected to the examination table-side connector 112, the reception-side cable 102 is stored in the cable storage part 106 by pulling in the examination table-side cable 104 via the cable drive mechanism 100, and the reception-side cable 102 is fed out from the cable storage part 106 by feeding out the examination table-side cable 104, so that it is possible to eliminate the cable entanglement problem.

In the embodiment, the interlocking configuration is described in which the operation of storing the reception-side cable 102 via the cable drive mechanism 100 and the operation of moving the top plate 18 into the bore 14 via the top plate drive mechanism 24 are performed by one top plate advancement operation of the top plate operation unit 132, but the present invention is not limited to this. For example, the cable drive mechanism 100 may be driven independently of the top plate operation unit 132 by separately providing a cable operation unit that operates only the cable drive mechanism 100. In this case, the top plate operation unit 132 operates only the top plate drive mechanism 24.

As described above, by providing the top plate operation unit 132 that operates only the top plate drive mechanism 24 and the cable operation unit that operates only the cable drive mechanism 100, the following operations can be performed. That is, it is possible to stagger the start times of the operation of moving the top plate 18 and the operation of storing the reception-side cable 102.

For example, in a case in which the extra length of the reception-side cable 102 is longer than a specified length, the operation of storing the reception-side cable 102 is started prior to the operation of moving the top plate 18. As a result, it is possible to effectively eliminate the above-described cable entanglement problem.

In addition, in a case in which the extra length of the reception-side cable 102 is shorter than the specified length, the operation of moving the top plate 18 is started prior to the operation of storing the reception-side cable 102. As a result, it is possible to prevent excessive tension from being applied to the reception-side cable 102.

That is, in the present form, the start time of each of the operation of moving the top plate 18 and the operation of storing the reception-side cable 102 is adjusted based on elements related to the cable entanglement problem, such as the extra length of the reception-side cable 102, the movement speed of the top plate 18, and the storage speed of the reception-side cable 102, so that the cable entanglement problem does not occur.

Further, the top plate 18 is moved to a position immediately before the extra length of the reception-side cable 102 is entangled, and then the movement of the top plate 18 is temporarily stopped, and the extra length of the reception-side cable 102 is stored in the cable storage part 106 at the stopped position. Thereafter, the top plate 18 is moved to the imaging position. In the present form, such an operation can also be performed.

On the other hand, in the embodiment, the MRI apparatus 10 in which the cable drive mechanism 100 is provided at one place on the top plate 18 has been described, but it is preferable that the cable drive mechanism 100 is disposed at each of positions corresponding to four corner portions of the top plate 18. As a result, it is possible to respond to the storage of the cable other than the reception-side cable 102.

### Modification Example

Hereinafter, some modification examples of the present invention will be described.

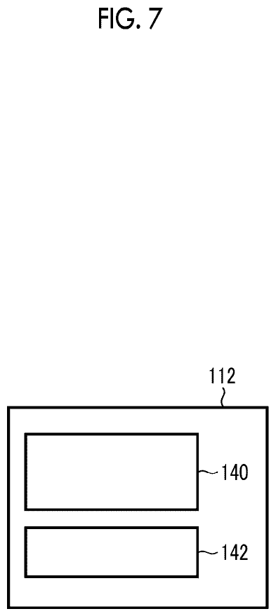

FIG. 7 is a schematic diagram showing a front side of the examination table-side connector 112. As shown in FIG. 7, the examination table-side connector 112 includes a connector connection area 140 to be connected to the reception-side connector 110, and a cable fixing area 142 to which a cable fixing member is attached.

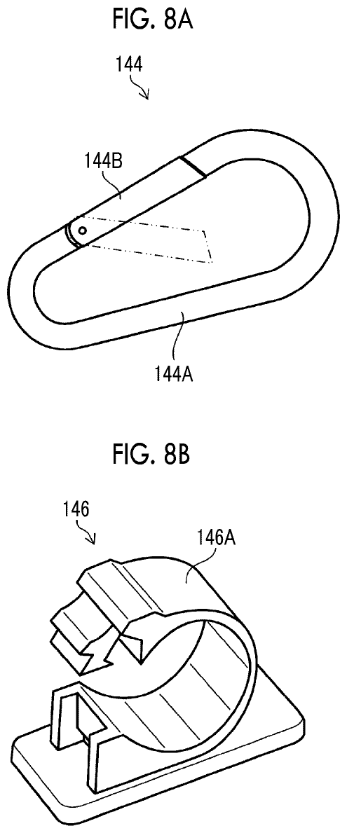

In the cable fixing area 142, for example, a cable fixing member 144 shown in FIG. 8A or a cable fixing member 146 shown in FIG. 8B is attached. In addition, a cable member (for example, a cable for an electro cardio gram (ECG) device, a cable for other sensors, and a tube for drip infusion) other than the reception-side cable 102 is attachably and detachably fixed to the cable fixing member 144 or the cable fixing member 146.

Here, the cable fixing member 144 shown in FIG. 8A is a carabiner-type openable/closable fixing member, and includes a C-shaped body part 144A and a pin-shaped opening/closing part 144B that is openably and closably provided on the body part 144A. The cable member is passed through the body part 144A and fixed. In this case, it is preferable to open and close the opening/closing part 144B with one touch to hook and fix the cable member to the body part 144A.

In addition, the cable fixing member 146 shown in FIG. 8B includes a C-type cable clip 146A. It is preferable that the cable member is passed through the cable clip 146A and fixed.

In this way, by providing the cable fixing area 142 in the examination table-side connector 112, and fixing the cable member other than the reception-side cable 102 to the cable fixing member 144 or the cable fixing member 146 attached to the cable fixing area 142, the cable member can be stored in the cable storage part 106 together with the reception-side cable 102.

The cable member has a sufficient length for attachment to the subject 20. It is preferable to store this cable member in the cable storage part 106 in order to eliminate the cable entanglement problem.

In the related art, a technician (operator) manually organizes the reception-side cable so that the reception-side cable does not get entangled in moving the top plate. In some cases, such as a head coil, there are a plurality of cables, and in addition to the coil cable, there are also a plurality of cables such as a drip infusion tube and an EGC measurement cable. The technician needs to set up a device and a patient and organize the cables while paying attention to a condition of the patient and MRI contraindicated articles such as metals, which places a heavy burden on the technician. Therefore, by adopting the connector configuration of the present example (see FIG. 7), it is possible to reduce the burden on the technician.

Next, a cable storage operation stop unit will be described. The cable storage operation stop unit has a function of forcibly stopping the cable storage operation via the cable drive mechanism 100. The cable storage operation stop unit is an example of a cable storage operation stop part according to the present invention.

There are several examples (Examples 1 to 5) of the cable storage operation stop unit.

### Example 1: Cable Length of Each Cable is Stored in Advance

The MRI apparatus 10 can recognize which coil is connected based on imaging order information and the connector connection of the coil. By holding information (ID or type of the coil or the like) on each coil and cable length information of each coil in association with each other, the cable length of the coil used in the examination is identified to control the cable pull-in amount.

In Example 1, the cable storage operation is not forcibly stopped, but it is possible to prevent a problem (such as excessive tension) that occurs in relation to the cable storage operation.

### Example 2: Tension of Cable is Detected

It is detected that an excessive tension is applied to the cable based on a motor load current during the cable storage operation, and the cable storage operation is forcibly stopped.

### Example 3: Cable State is Detected With Camera

At least one camera is provided at a position where a cable state on the examination table can be imaged, such as on a ceiling or a gantry, and the cable state (for example, the extra length of the cable) is detected with the camera, and the cable storage operation is forcibly stopped, for example, in a case in which there is no extra length left.

### Example 4: Marking is Provided on Cable, and Marking is Detected With Sensor

A marking is provided on the cable, the marking is detected by a detection unit such as a photosensor, and a cable storage amount and a storage stop position are detected based on information on the marking.

The cable storage operation is forcibly stopped in a case in which the cable storage amount and the storage stop position exceed a specified amount and a specified position. The marking on the cable may be provided only at the stop position, or may be provided at regular intervals to detect the storage amount.

### Example 5: A Plurality of Examples 1 to 4 Described Above are Combined

A plurality of Examples 1 to 4 described above may be combined. This makes it possible to cope with a case in which any of the cable storage operation stop units is not operated due to a malfunction. For example, control of Example 1 is normally performed, but control of Example 2 is also possible in preparation for the use of a coil without cable length information.

Next, a modification example in which the cable storage operation is manually executed will be described.

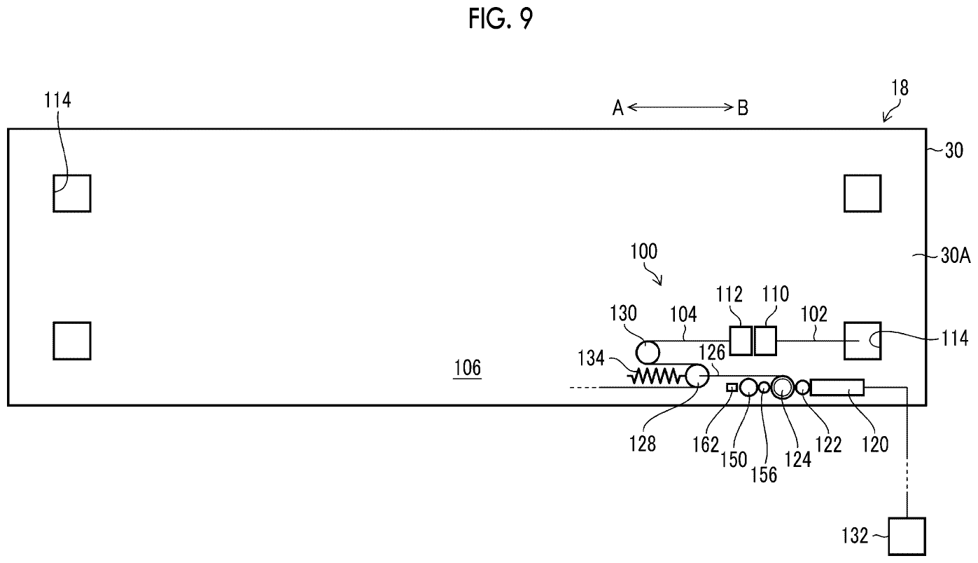

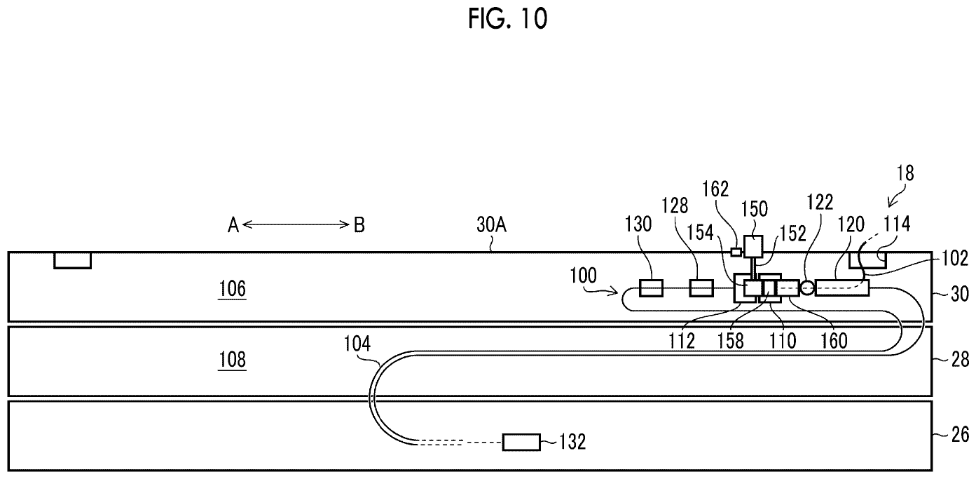

FIG. 9 is a top view of the cable drive mechanism 100 showing a handle 150 that manually drives the cable drive mechanism 100, and FIG. 10 is a side view of the cable drive mechanism 100 shown in FIG. 9.

In the description of FIGS. 9 and 10, members that are the same as or similar to the members of the cable drive mechanism 100 shown in FIGS. 4 and 5 are denoted by the same reference numerals, and the description thereof will be omitted. In addition, although the motor 120 is shown in FIGS. 9 and 10, the cable drive mechanism 100 of the present example is described as being driven by the handle 150.

As shown in FIGS. 9 and 10, the handle 150 is provided on the upper surface 30A of the upper top plate 30. A shaft 152 of the handle 150 is inserted into the internal space of the upper top plate 30, and a gear 154 is fixed to a lower end part of the shaft 152. The gear 154 is meshed with a gear 158 of an electromagnetic clutch 156, and the gear 158 is meshed with a gear 160 of the geared roller 124.

The electromagnetic clutch 156 is released by operating a release button 162. The release button 162 is disposed on the upper surface 30A of the upper top plate 30.

In a case in which the reception-side cable 102 is stored in the cable storage part 106, the geared roller 124 is rotated by manually rotating the handle 150 to wind the drive belt 126 around the geared roller 124. As a result, as described above, the reception-side cable 102 can be stored in the cable storage part 106.

After the imaging is ended, the electromagnetic clutch 156 is released by operating the release button 162 after the top plate 18 is pulled out to some extent (to the extent that a risk of the reception-side cable 102 being pulled into a gap between the gantry 12 and the examination table 16 is eliminated). As a result, the reception-side cable 102 is automatically fed out.

The handle 150 is in a semi-locked state by the electromagnetic clutch 156. That is, the handle 150 is free in a rotation direction in which the reception-side cable 102 is stored, but is locked in a rotation direction in which the reception-side cable 102 is fed out. Therefore, the electromagnetic clutch 156 is released in conjunction with the operation of pulling out the top plate 18. As a result, the above-described lock is released, and the reception-side cable 102 is automatically fed out by the biasing force of the spring 134 and returns to the original state.

In addition, after the imaging is ended, the top plate 18 may be automatically pulled out from the imaging position and the reception-side cable 102 may be automatically fed out from the cable storage part 106 without operating the top plate operation unit 132. In this case, in a case in which a controller that integrally controls the entire MRI apparatus 10 detects the end of the imaging, the controller controls the top plate drive mechanism 24 to automatically pull out the top plate 18 and release the electromagnetic clutch 156.

Next, the lock mechanism of the examination table-side connector 112 will be described.

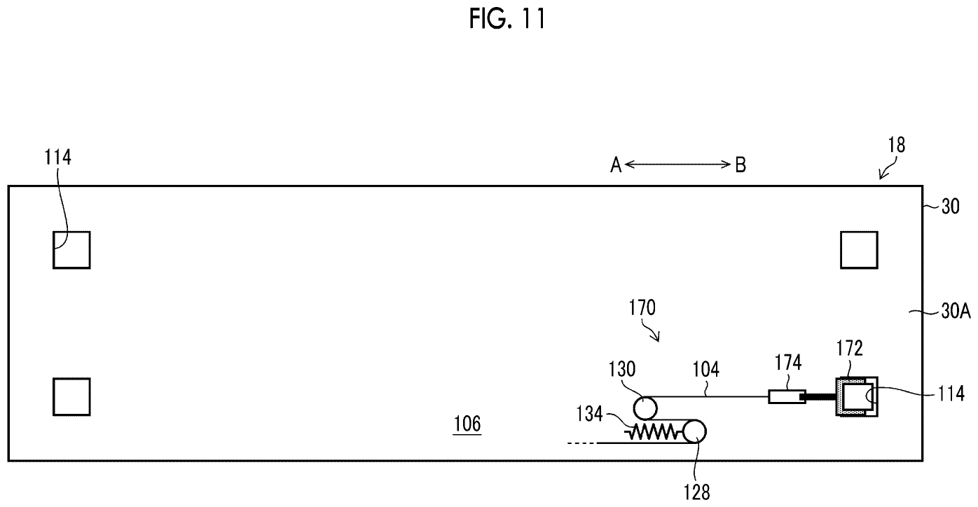

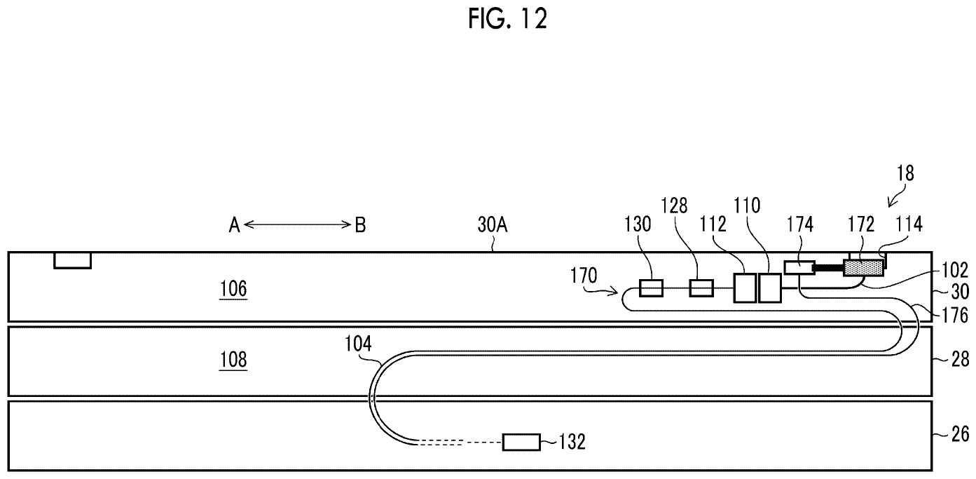

FIG. 11 is a top view showing a state in which entry of the examination table-side connector 112 into the cable storage part 106 is restricted (locked) by a lock mechanism 170. FIG. 12 is a side view of FIG. 11.

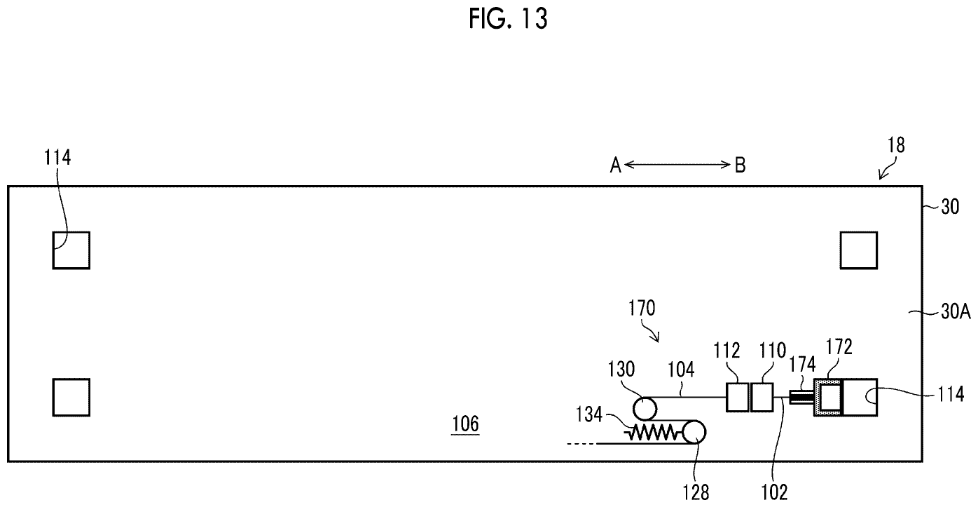

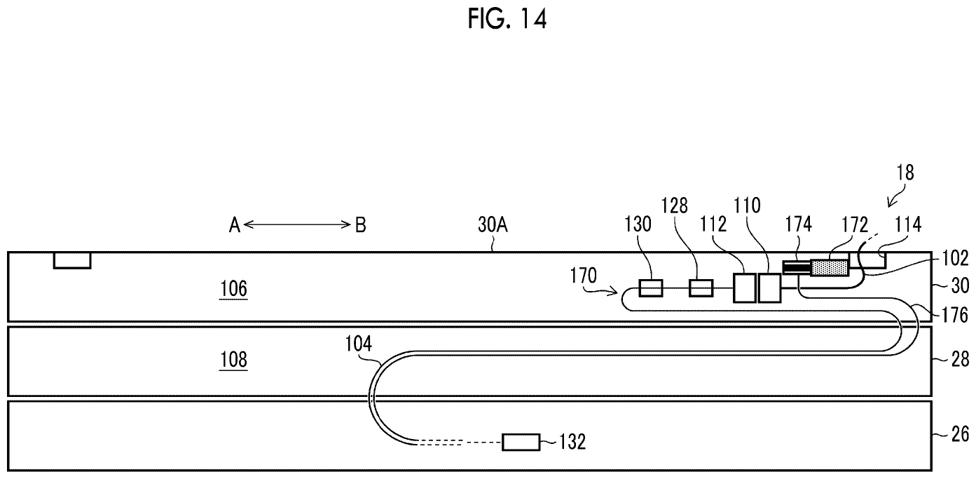

FIG. 13 is a top view showing a state in which the entry of the examination table-side connector 112 into the cable storage part 106 is released by the lock mechanism (170). FIG. 14 is a side view of FIG. 13.

As shown in FIGS. 11 to 14, in the lock mechanism 170 of the present example, a stopper member 172 is provided in the cable hole 114. The stopper member 172 is configured in a U-shape with a rectangular opening portion formed in a center portion thereof.

As shown in FIGS. 11 and 12, in a case in which the stopper member 172 is moved to advance downward of the cable hole 114, a part of the cable hole 114 is closed by the stopper member 172. Then, the examination table-side connector 112 abuts on the stopper member 172 to restrict the examination table-side connector 112 from entering the cable storage part 106. That is, the opening portion of the stopper member 172 is formed to be smaller than the examination table-side connector 112.

In addition, as shown in FIGS. 13 and 14, in a case in which the stopper member 172 is moved to retract in the direction of the arrow A from below the cable hole 114, the restriction by the stopper member 172 is released, so that the entry of the examination table-side connector 112 into the cable storage part 106 is allowed.

The stopper member 172 is moved to advance and retract by a known solenoid coil device 174. The solenoid coil device 174 is connected to a power supply unit (not shown) via a power control cable 176.

In a case in which a current from the power supply unit is supplied to the solenoid coil device 174, the stopper member 172 is moved from the retracted position of FIGS. 13 and 14 in the direction of the arrow B against a biasing force of a return spring (not shown) of the solenoid coil device 174, and is moved to the advance position of FIGS. 11 and 12.

In addition, in a case in which the power supply is stopped, the stopper member 172 is moved from the advance position of FIGS. 11 and 12 in the direction of the arrow A by the biasing force of the return spring of the solenoid coil device 174, and is moved to the retracted position of FIGS. 13 and 14.

By providing such a lock mechanism 170 on the top plate 18, it is possible to allow the entry of the reception-side cable 102 into the cable storage part 106 only in a case of being necessary (for example, in a case in which the top plate 18 is moved). In other words, it is possible to restrict the entry of the reception-side cable 102 into the cable storage part 106 in a case of being unnecessary (for example, in a case in which the RF coil 22 is attached).

Although the embodiment of the magnetic resonance diagnostic apparatus according to the present invention has been described above, the present invention may be improved or modified in various aspects without departing from the gist of the present invention.

### Explanation of References

10: MRI apparatus
16: examination table
18: top plate
20: subject
22: RF coil
24: top plate drive mechanism
100: cable drive mechanism
102: reception-side cable
104: examination table-side cable
106: cable storage part
108: cable storage part
110: reception-side connector
112: examination table-side connector
120: motor
132: top plate operation unit
144: cable fixing member
146: cable fixing member
150: handle

What is claimed is:

1. A magnetic resonance diagnostic apparatus comprising:
a reception coil that is attached to a subject to be imaged, and receives a magnetic resonance signal generated in the subject;
a first cable that is connected to the reception coil and has a first connector;
an examination table on which the subject is placed;
a cable storage part that is provided in the examination table and stores the first cable;
a second cable that is stored in the cable storage part and has a second connector connected to the first connector; and
a cable drive mechanism that pulls in or feeds out the second cable in the cable storage part,
wherein, in a state in which the first connector is connected to the second connector, the first cable is stored in the cable storage part by pulling in the second cable via the cable drive mechanism, and the first cable is fed out from the cable storage part by feeding out the second cable.

2. The magnetic resonance diagnostic apparatus according to claim 1, further comprising:
an operation unit that is operable by an operator,
wherein the cable drive mechanism is driven in a case in which the operation unit is operated.

3. The magnetic resonance diagnostic apparatus according to claim 2,
further comprising:
an examination table moving unit that moves the examination table to an imaging position,
wherein the examination table moving unit is driven by the operation of the operation unit.

4. The magnetic resonance diagnostic apparatus according to claim 3,
wherein the cable drive mechanism and the examination table moving unit are interlocked with each other by a single operation of the operation unit.

5. The magnetic resonance diagnostic apparatus according to claim 4,
wherein, in a case in which the operation unit is operated, an operation of storing the first cable in the cable storage part via the cable drive mechanism and an operation of moving the examination table to the imaging position are performed.

6. The magnetic resonance diagnostic apparatus according to claim 1,
wherein the cable storage part is provided on an outer peripheral part of the examination table.

7. The magnetic resonance diagnostic apparatus according to claim 1, further comprising:
a motor that stores the first cable in the cable storage part,
wherein the first cable is stored in the cable storage part by a driving force of the motor.

8. The magnetic resonance diagnostic apparatus according to claim 1,
wherein the second connector includes a cable fixing member that is capable of fixing a cable member different from the first cable.

9. The magnetic resonance diagnostic apparatus according to claim 7, further comprising:
a cable storage operation stop part that forcibly stops a storage operation of storing the first cable in the cable storage part.

10. The magnetic resonance diagnostic apparatus according to claim 2,
wherein the operation unit includes a handle that manually drives the cable drive mechanism.

11. The magnetic resonance diagnostic apparatus according to claim 3,
wherein the first cable is automatically fed out from the cable storage part in conjunction with an operation of pulling out the examination table from the imaging position via the operation unit.

12. The magnetic resonance diagnostic apparatus according to claim 3,
wherein after an examination is ended, the examination table is automatically pulled out from the imaging position, and the first cable is automatically fed out from the cable storage part.
